(19) United States
(12) Patent Application Publication
(10) Pub. No.: US 2008/0260601 A1
(43) Pub. Date: Oct. 23, 2008

(54) Uv Sterilizing Wand

(76) Inventor: Donald E. Lyon, Hanwell (CA)

Correspondence Address: SMART AND BIGGAR, 438 UNIVERSITY AVENUE, SUITE 1500 BOX 111, TORONTO, ON, M5G2K8, CA

(21) Appl. No.: 12/092,681

(22) PCT Filed: Nov. 3, 2005
(86) PCT No.: PCT/CA05/01688

§ 371 Date: May 5, 2008

(51) Int. Cl. A61L 9/00

(52) U.S. Cl. 422/186.3

(57) ABSTRACT

A wand-type Ultra-Violet (UV) radiation sterilizing wand includes a housing having an aperture and a source of UV radiation mounted within the housing positioned for emitting UV radiation through the aperture. Advantageously, the lamp chamber within which the source of UV radiation is mounted has an ingress and an egress so that an air flow may be promoted through the lamp chamber to cool the lamp and, thereby, allow for a lamp of higher UV power output than would otherwise be practical.

### FIELD OF THE INVENTION

The present invention relates to sterilization using Ultra-Violet (UV) radiation and, more particularly, to a wand-type UV sterilizer.

### BACKGROUND

The anti-microbial properties of UV-C light (Ultraviolet light-C band) are well-known to scientists and have been used since the 1930's to kill germs containing DNA and RNA (including bacteria, viruses, fungi and mold). UV-C light is invisible to the human eye. While UV-C light is invisible, given sufficient intensity and exposure, UV-C light can kill most of the germs responsible for causing disease in humans and animals. UV-C light can destroy the DNA and/or RNA (genetic material) of pathogens (disease-causing bacteria, viruses, mold, etc.). Once the DNA in a pathogen has been destroyed, the pathogen is either killed or deactivated; the pathogen can no longer function properly; and the pathogen can no longer reproduce.

Box-type UV sterilizers are well known for use in sterilizing all manner of objects including contact lenses, combs and safety goggles. Often only a single source of radiation is employed and, as such, there are often areas on an object to be sterilized that are shadowed from the UV radiation produced from the single source. Furthermore, the object to be sterilized is often required to rest on a support during the sterilization process. If the support is not transparent to the UV radiation, the support also contributes to shadowing the object to be sterilized from the UV radiation.

### SUMMARY

A germicidal wand helps eliminate disease-causing bacteria, viruses and fungi in a given environment. The wand-type Ultra-Violet (UV) radiation sterilizer allows a user to control the location of application of UV radiation and, thereby, sterilize areas on an object that may be shadowed when using a device with a fixed radiation source. The sterilizing wand includes a housing having an aperture and a source of UV radiation mounted within the housing positioned for emitting UV radiation through the aperture. Advantageously, the lamp chamber within which the source of UV radiation is mounted has an ingress and an egress so that an air flow may be promoted through the lamp chamber to cool the lamp and, thereby, allow for a lamp of higher UV power output than would otherwise be practical.

In accordance with an aspect of the present invention there is provided a sterilizing wand. The sterilizing wand includes a housing having a lamp chamber, the lamp chamber having an ingress and an egress in communication with an exterior of the housing, and an aperture in communication with the lamp chamber, the aperture positioned between the ingress and the egress. The sterilizing wand also includes a pane of filtering glass for covering the aperture, a C-band Ultra-Violet radiation source, mounted in the lamp chamber between the ingress and the egress, for emitting C-band Ultra-Violet radiation through the pane of filtering glass and an impellor installed in the lamp chamber for promoting a flow of air into the ingress from the exterior of the housing, past the C-band Ultra-Violet radiation source and but of the egress.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate example embodiments of this invention:

FIG. 1A illustrates a rear elevation view of a sterilizing wand according to an embodiment of the present invention;

FIG. 1B illustrates a rear elevation view of the sterilizing wand of FIG. 1A with an aperture cover removed to expose a pair of bulbs; and

FIG. 2 illustrates a front elevation view of the sterilizing wand of FIG. 1;

FIG. 3 illustrates a side sectional view of the sterilizing wand of FIG. 1;

FIG. 4 illustrates a bottom plan view of the sterilizing wand of FIG. 1;

FIG. 5A illustrates an LED display of the sterilizing wand of FIG. 1 in a Standby mode;

FIG. 5B illustrates the LED display of the sterilizing wand of FIG. 1 in a Safety mode;

FIG. 5C illustrates the LED display of the sterilizing wand of FIG. 1 in a Basic mode; and

FIG. 5D illustrates the LED display of the sterilizing wand of FIG. 1 in a Continuous mode.

### DETAILED DESCRIPTION

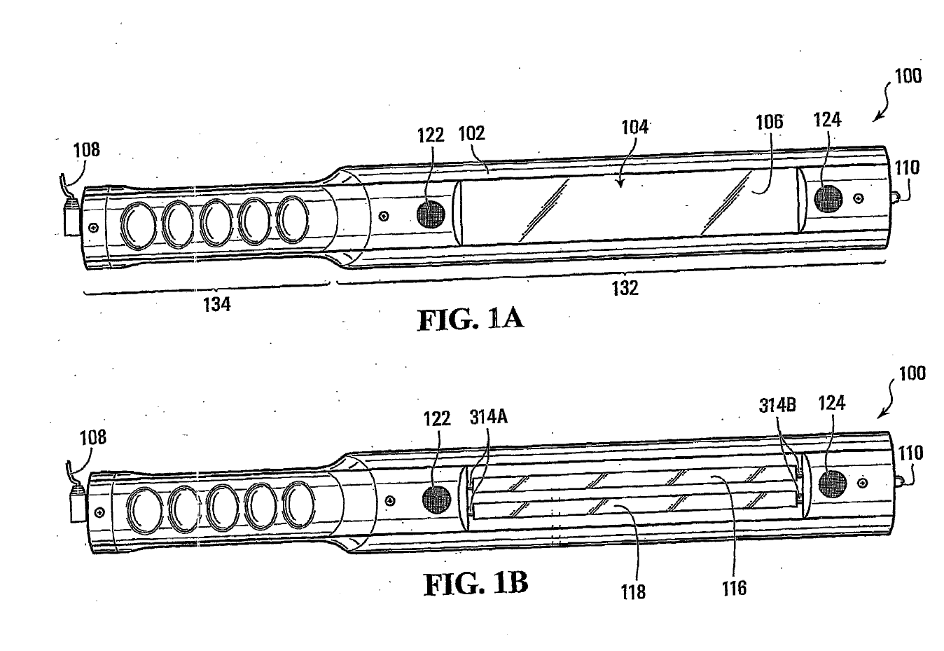

As illustrated in FIG. 1A, a sterilizing wand 100 includes a housing 102 with an aperture 104. The housing 102 may, for instance, be formed of Acrylonitrile Butadiene Styrene (ABS) plastic. Alternatively, the housing 102 may be formed of a plastic suitable for a CBRN (chemical/biological/radiological/nuclear) safety rating. Mounted within the aperture 104 may be a pane of filtering glass 106. The housing 102 of the sterilizing wand 100 may be considered to have an active portion 132 and a handle portion 134.

The sterilizing wand 100 is illustrated in FIG. 1B without the pane of filtering glass 106. As such, a pair of lamps, normally hidden by the pane of filtering glass 106, are in evidence. In particular, a lamp chamber in the housing 102 may house a C-band Ultra-Violet (UV-C) lamp 116 and an A-band UV (UV-A) black light lamp 118. The UV-C lamp 116 may be, for instance, an industry standard G6T5 germicidal lamp. Generically, a suitable standard lamp may be described by GXTM, where G indicates that the lamp is germicidal, X is representative of an electrical input power rating in Watts and M is representative of the lamp diameter in ⅛ inch units. Accordingly, a G6T5 lamp has a 6 Watt power rating and a roughly ⅝ inch diameter. As will be clear to a person of ordinary skill in the art, the C-band Ultra-Violet radiation source need not be a GXTM lamp. For instance, UV-C light emitting diodes (LEDs) may be used.

Material for the pane of filtering glass 106 may be selected to be transparent to UV-C light and to minimize the transmission 185 nm by-product light from the UV-C lamp 116. The 185 nm by-product light is known to cause the production of ozone.

In an alternative embodiment, the lamp chamber in the housing 102 houses two UV-C lamps and a UV-A LED 110 is installed at the end of the active portion 132 of the sterilizing wand 100.

The sterilizing wand 100 is illustrated in FIGS. 1A and 1B to include an ingress 122 to the lamp chamber as well as an egress 124 from the lamp chamber.

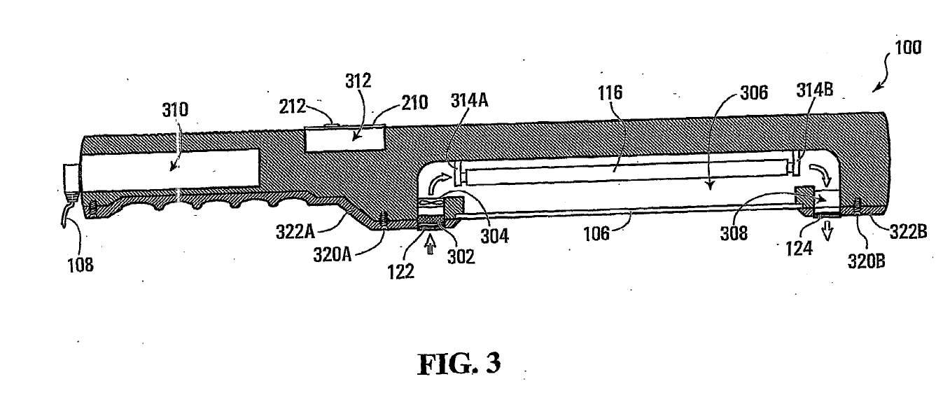

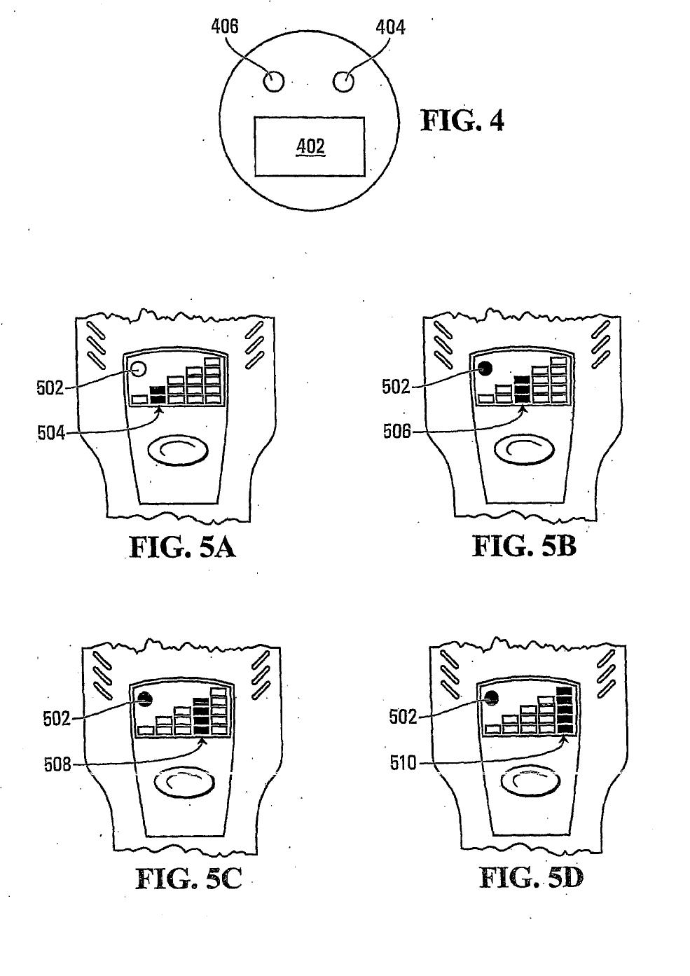

To provide power to the lamps 116, 118, an alternating current (AC) adapter may be attached to the sterilizing wand 100 via a primary power cord 108 with an end that is received by a receptacle on the bottom of the wand (see FIG. 4). It is contemplated that the AC adapter may be rated to provide 12V and 10W for a consumer version of the sterilizing wand 100 and 12V and 20W for an industrial version of the sterilizing wand 100. The industrial version of the sterilizing wand 100 may, as discussed above, have two UV-C lamps installed in the lamp chamber and a UV-A LED 110 on the exterior of the sterilizing wand 100.

An alternative power supply may be provided by a set of batteries (not shown), which may be installed within a battery compartment, say, in the handle portion 134 of the housing 102. For instance, the sterilizing wand 100 may be powered by eight AA batteries. The batteries may, for instance, be disposable alkaline or rechargeable, such as Lithium ion or Nickel Metal Hydride. A further alternative power supply may be provided by a battery belt, which could hold hot-swappable batteries and be worn by the user. Power may be supplied from the battery belt to the sterilizing wand 100 via a secondary power cord (not shown).

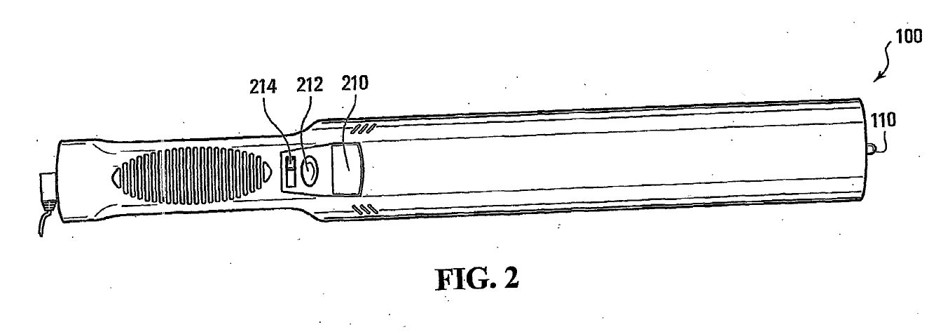

FIG. 2 illustrates a front elevation view of the sterilizing wand 100 of FIG. 1. Evident in FIG. 2 is a display 210, which may be used to display information about the operation of the sterilizing wand 100. Such information may, for instance, include on/off status of each of the lamps. Such information may, for instance, also include battery charge status. Also evident in FIG. 2 is a control button 212 that may be provided for activating the UV-C lamp 116 and a toggle switch 214 that may be provided for activating the UV-A black light lamp 118.

FIG. 3 illustrates a side sectional view of the sterilizing wand 100. The sectional view of FIG. 3 provides insight to a cooling and ozone removal mechanism. In particular, an impellor fan 304 is located in the lamp chamber 306. The Ingress 122 to the lamp chamber 306 is covered by a dust filter 302 while air flows out of the lamp chamber 306 via an ozone catalyst chamber 308 at the egress 124 of the lamp chamber 306.

A variety of manufacturers produce ozone catalysts. Ozone catalysts are most commonly based on manganese dioxide and operate by acting as a reaction site for conversion of O3 to O2. The ozone catalyst is simply a medium that air passes through.

Furthermore, the sectional view of FIG. 3 provides insight into the manner in which the pane of filtering glass 106 is held in place. In particular, a first hold-down plate 322A attaches to the housing 102 by a securing means, for example, by a first screw 320A. Similarly, a second hold-down plate 322B attaches to the housing 102 by a securing means, for example, by a second screw 320B.

The UV-C lamp 116 is illustrated in FIG. 3 as being supported by a first lamp support 314A and a second lamp support 314B. Typical lamp supports are known to be manufactured of hard plastics and ceramics and, as such are generally inflexible. It is recognized that use of the sterilizing wand 100 in an industrial environment may expose the sterilizing wand 100 and, consequently, the UV-C lamp 116 to sudden, jarring motion. Such motion in combination with inflexible lamp supports may lead to a frequent need to replace broken lamps. It is proposed herein, then, to incorporate shock absorbing qualities in the lamp supports 314A, 314B. For instance, the lamp supports 314A, 314B may be composed of a flexible material or may be connected to the housing 102 using a flexible mount.

In further evidence in the sectional view of FIG. 3 is a battery compartment 310 and an electronics-compartment 312.

As will be understood by a person of ordinary skill in the art, a controller, in the form of a microprocessor, may be installed in the electronics compartment 312 to perform many functions.

FIG. 4 illustrates the bottom of the sterilizing wand 100 of FIG. 1. In particular, the bottom has a battery door 402, which may be removed for installation and replacement of the batteries. The bottom also has a first power receptacle 404 for receiving a plug on the end of the primary power cord 108 from the AC adapter and a second power receptacle 406 for receiving a plug on the end of the secondary power cord from the battery belt.

The connection between the primary power cord 108 from the AC adapter and the first power receptacle 404 may be latched to prevent accidental disconnection of the primary power cord 108. Similarly, the connection between the secondary power cord and the second power receptacle 406 may be latched to prevent accidental disconnection of the secondary power cord.

In one embodiment, the sterilizing wand 100 has four different modes of operation: a “Standby” mode; a “Safety” mode; a “Basic” mode; and a “Continuous” mode.

In operation, a user may first don UV Resistant Safety Glasses. Subsequently, the user may insert the plug on the end of the primary power cord 108 into the receptacle 404 on the end of the sterilizing wand 100. The user may then plug the AC adapter into an electrical outlet.

The sterilizing wand 100 enters the Standby mode upon being plugged in to a power supply. To indicate to the user that the sterilizing wand 100 is receiving power, the sterilizing wand 100 may illuminate two LEDs 504 on the display 210 (see FIG. 5A). The sterilizing wand 100 may require approximately 60 seconds to warm up. Once warmed up and ready to be used, the sterilizing wand 100 may “beep” to notify the user of such readiness. A speaker (not shown) to emit such a beep may be located in the electronics compartment 312.

The user may rapidly press the control button 212 twice to enter Safety mode. Responsive to the control button 212 being pressed twice, the speaker may sound a beep and activate the UV-C lamp 116. To indicate to the user that the sterilizing wand 100 has entered Safety mode, the sterilizing wand 100 may illuminate three LEDs 506 oh the display 210 (see FIG. 5B). In addition, the sterilizing wand 100 may illuminate a further LED 502 on the display 210. The further LED 502 may be considered a “Run” light 502 and may be used to indicate to the user that the UV-C lamp 116 is activated. While the sterilizing wand 100 is in Safety mode and the UV-C lamp 116 is activated, the speaker may periodically, say, every ten seconds, sound a beep. Characteristic of the Safety mode, the sterilizing wand 100 may de-activate the UV-C lamp 116 after five minutes of operation and return to the Standby mode.

It is known that, in most cases, only 10 to 20 seconds of direct exposure to UV-C light will kill most household germs. As such, the user may use the periodic beep sounds to gauge the necessary exposure of, an object or area being sanitized. To de-activate the UV-C lamp 116 before the end of the five minutes period of operation, the user may press the control button 212 once.

The user may rapidly press the control button 212 three times to enter Basic mode. Responsive to the control button 212 being pressed three times, the speaker may sound a beep and activate the UV-C lamp 116. To indicate to the user that the sterilizing wand 100 has entered Basic mode, the sterilizing wand 100 may illuminate four LEDs 508 on the display 210 (see FIG. 5C). In addition, the sterilizing wand 100 may illuminate the Run light 502 on the display 210 to indicate to the user that the UV-C lamp 116 is activated. While the sterilizing wand 100 is in Basic mode, and the UV-C lamp 116 is activated, the speaker may periodically, say, every ten seconds, sound a beep. Characteristic of the Basic mode, the sterilizing wand 100 may de-activate the UV-C lamp 116 after two hours of operation and return to the Standby mode. To de-activate the UV-C lamp 116 before the end of the two hour period of operation, the user may press the control button 212 once.

The user may rapidly press the control button 212 four times to enter Continuous mode. Responsive to the control button 212 being pressed four times, the speaker may sound a beep and activate the UV-C lamp 116. To indicate to the user that the sterilizing wand 100 has entered Continuous mode, the sterilizing wand 100 may illuminate five LEDs 510 on the display 210 (see FIG. 5D). In addition, the sterilizing wand 100 may illuminate the Run light 502 an the display 210 to indicate to the user that the UV-C lamp 116 is activated. In contrast to the Safety and Basic modes of operation, while the sterilizing wand 100 is in Continuous mode and the UV-C lamp 116 is activated, the speaker may not sound a periodic beep, Characteristic of the Continuous mode, the sterilizing wand 100 may not de-activate the UV-C lamp 116 until the user presses the control button 212 once.

At any time that the UV-C lamp 116 is activated, the fan 304 may also be activated. The activated fan 304 draws air into the lamp chamber 306 through the dust filter 302 and forces the air past the lamps 116, 118 and through the ozone catalyst chamber 308. It is known that when the UV-C lamp 116 is operating, oxygen (O₂) in air nearby to the UV-C lamp 116 will be converted into ozone (O₃). The activation of the fan has multiple advantageous effects. The air drawn from outside of the sterilizing wand 100, at the ingress 122, can act to cool the UV-C lamp 116 and can act to flush the ozone created by the operation of the UV-C lamp 116 into the ozone catalyst chamber 308, where the ozone (O₃) may be converted to oxygen (O₂) before being expelled from the sterilizing wand 100 at the egress 124.

The UV output of the UV-C lamp 116 is temperature dependent and diminishes at higher temperatures. It has been found that optimum output is generally in the 40-50° C. range. As such, the controller may monitor the temperature of the lamp chamber 306 and control the fan 304 to attempt to maintain a temperature in the optimum range.

Furthermore, even though the pane of filtering glass 106 may be selected to minimize the transmission 185 nm by-product light from the UV-C lamp 116, some 185 nm by-product light will likely be emitted and, as such, ozone will likely be created outside of the sterilizing wand 100.

By drawing air into the lamp chamber 306, some of the ozone surrounding the sterilizing wand will also be drawn into the lamp chamber 306 and forced through the ozone catalyst chamber 308, where the ozone (O₃) may be converted to oxygen (O₂) before being expelled from the sterilizing wand 100.

For disinfecting an element of a bed, for example, a mattress, a quilt or a pillow, it is recommended that the bed element be vacuumed first. The sterilizing wand 100 may be held approximately eight inches (20 cm) above the surface of the bed element and may be moved sideways over one foot (30 cm) for 20 seconds. The location of the sterilizing wand 100 may then be adjusted so that a further one-foot-long portion of the bed element may be exposed to the radiation from the UV-C lamp 116. Exposure and position changing may be continued until the entire desired surface area of the bed element has been exposed. The same approach for disinfecting an element of a bed may be used for disinfecting an animal mattresses or enclosure.

Toothbrushes, razors and other personal hygiene implements may be disinfected through exposure at closed range (a few inches or less) for a minimum of 10 seconds.

For kitchen utensils, such as knives, utensils and chopping boards, the sterilizing wand 100 may be held at close range for a minimum of 10 seconds. Notably, the sterilizing wand should be used away from water sources.

For fruit, the sterilizing wand 100 may be held approximately 8 inches (20 cm) above target surfaces for 20 seconds of exposure. Notably, over-exposing fruit may change the taste.

For general bathroom germs and odors, the sterilizing wand 100 may be held approximately four inches (10 cm) above the surface of the toilet seat and slowly move the sterilizing wand 100 sideways over the entire surface for a total of 20 seconds. Care should be exercised so as not to immerse the sterilizing wand 100 in water.

To control airborne germs in the bathroom, the sterilizing wand 100 may be hung in the bathroom for one hour while the bathroom is unoccupied. For safety, the bathroom should be locked to prevent children or others from unsuspectingly entering the bathroom while disinfection is taking place.

To minimize or eliminate musty smells in rooms, the sterilizing wand 100 may be placed in Basic mode and left in the room for two hours, Again, the room should be locked.

Shoes may be exposed once a week at close range for five minutes to kill germs that cause athlete's foot. The soles of the shoes may be exposed more frequently. It has been recognized that the soles of shoes are a significant source of bacteria in the home.

Computer keyboards, doorknobs, telephones, etc. may be exposed at a distance of 8 inches (20 cm) for 10 seconds.

When in doubt, it has been found that effective disinfection can generally be achieved in 20 seconds at a distance of about 8 inches (20 cm).

The UV-A black light lamp 118 may be used as both a stain detector and a flashlight. In particular, the UV-A black light lamp 118 may be used to locate sources of pet odor, such as traces of urine, feces and vomit that may be invisible under normal light conditions.

In operation, it is recommended that the user darken the room in which stain detection is to take place. The UV-A black light lamp 118 may then be activated through use of the toggle switch 214 (see FIG. 2). The user may then hold the sterilization wand 100 above a surface on which stain detection is to take place. The user may recognize contamination as pale spots of glowing yellow. The user may then use the toggle switch 214 to de-activate the UV-A black light lamp 118 and press the control button 212 a number of times to activate the UV-C lamp 116 in a particular-operational mode.

As should be clear, while use of the UV-C lamp 116 of the sterilization wand 100 may kill the microorganisms that cause an odor associated with a stain that may be found using the UV-A black light lamp 118, the stain will not be removed.

Software executed by the microprocessor in the electronics compartment 312 may allow the microprocessor to monitor factors related to battery health, including such factors as current, voltage and battery temperature. Software executed by the microprocessor may allow the microprocessor to control the LEDs of the display 210. Software executed by the microprocessor may allow the microprocessor to provide an indication of a number of available batteries in a power supply setup wherein the batteries, either internal or external to the sterilizing wand 100, are hot-swappable. That where the sterilizing wand 100 may switch from receiving-power from a first set of batteries to receiving power from a second set of batteries, while in operation. Software executed by the microprocessor may allow the microprocessor to monitor lamp health, for instance, by monitoring the amount of current drawn by a lamp. Software executed by the microprocessor may also allow the microprocessor to monitor and report on the mode of operation by controlling the LEDs of the display 210. Software executed by the microprocessor may allow the microprocessor to monitor and report on the source of power, that is, whether the power is being drawn from batteries, from the first power receptacle 404 or from the second power receptacle 406.

Advantageously, the sterilizing wand 100 has a lightweight, convenient, hand-held design and eliminates bacteria, viruses, funguses, molds, etc., without harmful chemicals and leaves no chemical residue. Notably, moderate exposure to UV light has been found to be harmless to the materials that are being disinfected. In aspects of the present invention, the display 210 informs the user that the UV-C lamp 116 is activated.

Further advantageously, as produced by the action of the fan 304, the air flow past the UV-C lamp 116 provides a cooling effect that allows for the use of a more powerful UV-C lamp than may be used in conventional hand-held UV sterilizers. A lamp of higher UV power output may be seen to kill more germs in the same period of time than a lamp of lower UV power output. Alternatively, a lamp of higher UV power output may be seen to kill a fixed number germs faster than a lamp of lower UV power output. The air flow also allows for use of the ozone catalyst chamber 308 to reduce or remove the ozone before the air is expelled from the lamp chamber 306. Additionally, the fan 304 acts to bring air into the lamp chamber 306 from around the sterilizing wand 100. In the event that the use of the sterilizing wand has produced ozone in the air external to the sterilizing wand 100, the ozone catalyst chamber 308 may also reduce or remove the ozone in the external air.

Additionally, safety features of the sterilizing wand 100 include the pane of filtering glass 106, which may contain a broken lamp, and the automatic shutoff of the UV-C lamp 116 in two of the three operational modes.

Other modifications will be apparent to those skilled in the art and, therefore, the invention is defined in the claims.

I claim:

1. A sterilizing wand comprising:
a housing having:
a lamp chamber, said lamp chamber having an ingress and an egress in communication with an exterior of said housing; and
an aperture in communication with said lamp chamber, said aperture positioned between said ingress and said egress;
a pane of filtering glass for covering said aperture;
a C-band Ultra-Violet radiation source, mounted in said lamp chamber between said ingress and said egress, for emitting C-band Ultra-Violet radiation through said pane of filtering glass; and
an impellor installed in said lamp chamber for promoting a flow of air into said ingress from said exterior of said housing, past said C-band Ultra-Violet radiation source and out of said egress.

2. The sterilizing wand of claim 1 further comprising an ozone catalyst chamber, mounted at said egress of said lamp chamber, for converting ozone in said air flow to oxygen.

3. The sterilizing wand of claim 1 further comprising a dust filter, mounted at said ingress of said lamp chamber, for filtering particulate matter from said air flow before said air flow passes through said lamp chamber.

4. The sterilizing wand of claim 1 further comprising an A-band Ultra-Violet radiation source, mounted in said lamp chamber between said ingress and said egress, for emitting A-band Ultra-Violet radiation through said pane of filtering glass.

5. The sterilizing wand of claim 1 further comprising an A-band Ultra-Violet light emitting diode, mounted to said housing, for emitting A-band Ultra-Violet radiation.

6. The sterilizing, wand of claim 1 further comprising a second C-band Ultra-Violet radiation source, mounted in said lamp chamber between said ingress and said egress, for emitting C-band Ultra-Violet radiation through said pane of filtering glass.

7. The sterilizing wand of claim 1 further comprising a controller operable to, according to input from a user, activate said C-band Ultra-Violet radiation source.

8. The sterilizing wand of claim 7 wherein said controller is further operable to, according to input from a user, deactivate said C-band Ultra-Violet radiation source.

9. The sterilizing wand of claim 7 wherein said controller is further operable to deactivate said C-band Ultra-Violet radiation source after a predetermined time period.

10. The sterilizing wand of claim 9 further comprising a speaker, wherein said controller is further operable to cause said speaker to periodically emit a sound while said C-band Ultra-Violet radiation source is activated.

11. The sterilizing wand of claim 9 wherein said predetermined time period is around five minutes.

12. The sterilizing wand of claim 9 wherein said predetermined time period is two hours.

13. The sterilizing wand of claim 7 wherein said controller is further operable to monitor a temperature in said lamp chamber and control said impellor to obtain a temperature in a predetermined temperature range.

14. The sterilizing wand of claim 13 wherein said predetermined temperature range extends from around 40° Celsius to around 50° Celsius.

15. The sterilizing wand of claim 1 wherein said pane of filtering glass minimizes transmission of radiation around a 185 nm wavelength.

16. The sterilizing wand of claim 1 further comprising a lamp support for supporting said C-band Ultra-Violet radiation source.

17. The sterilizing wand of claim 16 further comprising a shock absorbing mount for connecting said lamp support to said housing.

18. The sterilizing wand of claim 1 wherein said C-band Ultra-Violet radiation source is a GXTM germicidal lamp.

19. A sterilizing wand comprising:
a housing having:
a lamp chamber, said lamp chamber having an ingress and an egress in communication with an exterior of said housing; and
an aperture in communication with said lamp chamber, said aperture positioned between said ingress and said egress;
a means for covering said aperture;
a means, mounted in said lamp chamber between said ingress and said egress, for emitting C-band Ultra-Violet radiation through said means for covering said aperture; and
a means, installed in said lamp chamber, for promoting a flow of air into said ingress from said exterior of said housing, past said means for emitting C-band Ultra-Violet radiation and out of said egress.
